(19) United States
(12) Patent Application Publication
(10) Pub. No.: US 2020/0038314 A1
(43) Pub. Date: Feb. 6, 2020

(54) PROCESS FOR PREVENTING OR SLOWING THE APPEARANCE OF UNATTRACTIVE SIGNS, GENERATED BY POLLUTANTS PRESENT IN THE ATMOSPHERE, ON THE SKIN, THE SCALP, THE HAIR OR THE MUCOUS MEMBRANES

(71) Applicants: SOCIETE D'EXPLOITATION DE PRODUITS POUR LES INDUSTRIES CHIMIQUES SEPPIC, PARIS (FR); BIOTECHMARINE, QUEMPER GUEZENNEC (FR)

(72) Inventors: Armelle BERGE, Castres (FR); Laetitia CATTUZZATO, CASTRES (FR); Erwan LE GELEBART, PLOUBAZLANEC (FR)

(21) Appl. No.: 16/338,620

(22) PCT Filed: Sep. 20, 2017
(86) PCT No.: PCT/FR2017/052521

§ 371 Date: Apr. 1, 2019

(30) Foreign Application Priority Data
Sep. 30, 2016 (FR) 1659394

(51) Int. Cl. A61K 8/9717; A61Q 19/00; A61Q 5/00

(52) U.S. Cl. CPC A61K 8/9717; A61Q 5/00; A61Q 19/00

(57) ABSTRACT

Process with the aim of preventing or slowing the appearance of unattractive signs, generated by the pollutants present in the atmosphere, on the skin, the scalp, the hair or the mucous membranes, or indeed of eliminating them, including a step of application of a cosmetic formulation for topical use, including at least one cosmetically acceptable excipient and an effective amount of at least one glycolic extract (GE) of a unialgal biomass of small multicellular macroalgae originating from the Florideophyceae class. The extract, for the use thereof in a therapeutic method for treating signs of irritation of the skin, the scalp or the mucous membranes, manifesting as redness, sensations of stinging and/or itching, signs of deterioration of the hair, manifesting as an increase in the porosity thereof, the dulling thereof, and/or the weakening thereof with regard to mechanical stresses.

The subject of the present invention is the use of an extract of unialgal biomass of small multicellular macroalga cells, as an anti-pollution agent for protecting human skin against the unattractive effects of pollution, and also a process for cosmetic treatment of human skin aimed at preventing unattractive effects of pollution, by application of a topical composition comprising an extract of unialgal biomass of small multicellular macroalga cells.

The effects of pollution on human health constitute health problems encountered on a worldwide scale. Indeed, urban environments are regularly exposed to various aerial and atmospheric pollutants, the action of which can prove to be harmful to the health of human beings, by deteriorating the functioning of certain organs such as the lungs and the eyes for example. By constituting a barrier between the human body and the outside environment, the keratin materials constituting the human body, and more particularly the skin, the mucous membranes and the hair, are in contact with these aerial and atmospheric pollutants, resulting in visible external effects characterized by the appearance and increase of wrinkles, by a dull complexion, by a lack of uniformity of the complexion (dyschromia phenomenon), by the appearance and increase of pigment spots (hyperpigmentation phenomenon) or else by a modification and a deterioration of the effectiveness of the cutaneous barrier.

The systemic effects of exposure to various types of pollutants are described in the literature, and the effects at the cutaneous level are being increasingly studied. Among the most harmful pollutants, mention may be made of toxic gases such as carbon monoxide, nitrogen oxides such as nitrogen dioxide, sulfur oxides such as sulfur dioxide, ozone; heavy metals, for instance cobalt, mercury, cadmium and nickel, inside pollutants, for instance volatile organic compounds (or COVs) and cigarette smoke, atmospheric particles which are microscopic solids in suspension in the Earth's atmosphere, for instance the microparticles identified under the acronyms PM2.5 and PM10 [E. Drakaki, C. Dessinioti and C. V. Antoniou, Frontiers in Environmental Science, 2, 1-6 (2014)].

These pollutants are known to cause visible external negative effects on the skin, resulting in or contributing to the appearance of wrinkles, of a dull complexion, of a lack of uniformity of the complexion (dyschromia phenomenon), of pigment spots and also of skin disorders such as psoriasis and atopical dermatitis [M A Lefebvre, D M Pham, B Boussouira, D Bernard, C Camus and Q L Nguyen, International Journal of Cosmetic Science, 37, 329-38 (2015)], [M A Lefebvre, D M Pham, B Boussouira, H Qiu, C Ye, X Long, R Chen, W Gu, A Laurent and Q L Nguyen, International Journal of Cosmetic Science, 38, 217-23 (2016)], [A. Vierkötter, Hautarzt, 61, 538-9 (2010)], [Y S Yang, H K Lim, K K Hong, M K Shin, J W Lee, S W Lee and N I Kim, Annals of Dermatology, 26, 11-16 (2014)].

At the current time, four biological components are supposed to be involved in the appearance of these visible external negative effects and these disorders [S. E. Mancebo and S. Q. Wang, Journal of European Academy of Dermatology and Venerology, 29, 2326-2332 (2015)], [N D Magnani, X M Muresan, G Belmonte, F Cervellati, C Sticozzi, A Pecorelli, C Miracco, T Marchini, P Evelson and G Valacchi, Toxicological Sciences, 149, 227-36 (2016)]:
the oxidizing component: polluting agents are thought to cause an increased production of free radicals by skin cells, resulting in the generation of carbonylated proteins, of peroxidized lipids or else in the appearance of DNA damage, and, in parallel, a decrease in enzymatic and non-enzymatic antioxidant systems is observed;
the inflammatory component: the effects of polluting agents are thought to bring about an induction of an inflammatory reaction cascade;
the activation of a hydrocarbon-specific receptor (AhR);
the degradation of the skin microflora.

These pathways are generally activated synergistically by the combination of the various polluting agents.

It is clearly apparent that there is an increasingly great demand to develop and produce topical compositions comprising chemical substances and compositions which make it possible to treat and prevent the unattractive effects of polluting agents on human skin. Said chemical substances and compositions may act according to various approaches:
by protecting the skin mechanically, that is to say by isolating it from polluting agents or, more realistically, by limiting its exposure to said polluting agents through the application to the skin of a “barrier” material, for example a topical composition having a film-forming effect;
by protecting the skin chemically, that is to say by applying to the skin one or more chemical substances or compositions which act as a chemical barrier, for instance chemical substances or compositions which have antioxidant properties, which supplement the action of the antioxidant defences intrinsically present on the skin, and/or heavy-metal-chelating properties, which are capable of capturing and trapping said polluting agents, and more particularly heavy metals, and consequently reducing or eliminating the oxidative effect of said polluting agents on the skin.
by protecting the skin biologically, that is to say by applying to the skin one or more chemical substances or compositions which act on the cells of the skin epidermis to increase their capacity to reduce or eliminate the harmful effects of polluting agents, for instance reduce or eliminate the oxidizing or inflammatory effects of said polluting agents.

Among the chemical substances and compositions capable of being incorporated into cosmetic compositions for topical use for chemically protecting the skin as described above, mention may be made of chelating agents, for instance ethylenediaminetetraacetic acid (or EDTA), the pentasodium salt of ethylenediaminetetramethylenephosphonic acid, and N,N′-bis-(3,4,5-trimethoxybenzyl)ethylenediamine or a salt, metal complex or ester thereof; phytic acid, tea extracts and more particularly green tea extracts; extracts of water hyacinth (Eichornia crassipes); and the water-soluble fraction of corn sold by the company Solabia under the trade name Phytovityl™.

In order to combat these negative effects of polluting agents on the keratin surfaces of the human body, the European patent application published under number EP 557 042 A1 discloses the use of metallothioneins against the effect of heavy metals and of sphingolipids for protecting more particularly the skin and the hair.

The European patent application published under number EP 1 230 914 A1 describes the use of a native or modified starch, in particular chosen from corn, rice, cassava, potato, wheat, sorghum and pea starches, and also the combination of said starches with a silicone gum, for mechanically protecting human keratin materials against the unattractive effects of polluting agents, and more particularly polluting agents which are in the form of atmospheric particles.

Ingredients of marine origin are of interest for cosmetic finished products by virtue of their natural origin, but can pose problems of odor, stability, efficacy, difficulty and non-reproducibility of formulated pharmaceutical forms, limiting their use. The marine resource is extremely large, and yet a whole section of marine diversity remains unexplored and/or unexploited.

Algae are chlorophyllous photosynthetic organisms which live in water or very moist environments. They can grow in seawater, freshwater, brackish water in stagnant, churned or turbulent environments. Algae may be unicellular or multicellular, they may be brown, green or red and are classified according to characteristics of cytological and biochemical type. These organisms play an important role on the global scale since they constitute the basis of trophic networks and they are involved in the production of atmospheric oxygen and in carbon dioxide fixation.

Macroalgae are eukaryotic multicellular algae, most often visible to the naked eye and often described as macrophytes. They can reproduce sexually or asexually; in some species, these two methods of reproduction follow on from one another during the life cycle. The life cycle may comprise one generation, or an alternation of two generations or three generations. The term respectively used will be monogenetic, digenetic or trigenetic cycles. Depending on the type of life cycle, the species can be in the gametophyte, sporophyte, carposporophyte or tetrasporophyte form. Over the course of a cycle, these various life forms may have the same morphologies and therefore be isomorphic, or have different morphologies and therefore be heteromorphic. The difference in morphology may be very significant, making it impossible to attach two life forms to the same species for an uninformed person.

This difference may also be marked by the size of the alga; one life form may be macroscopic and another life form may be microscopic. For example, the sporophyte of Undaria pinnatifida is close to a meter in size, whereas the gametophyte of this alga is a few tens of micrometers in size. There are approximately two thousand species of brown algae, seven thousand species of red algae and one thousand seven hundred species of green algae.

The macroalgae are a few tens of micrometers in size in the case of algae of the Acrochaetium genus to about a hundred meters for the Macrocystis pyrifera species.

The class of red algae, also known as Florideophyceae, is part of the Rhodophyte branch. The red algae class Florideophyceae comprises the orders Acrochaetiales, Acrosymphytales, Ahnfeltiales, Balbianiales, Balliales, Batrachospermales, Bonnemaisoniales, Ceramiales, Colaconematales, Corallinales, Entwisleiales, Florideophyceae incertae sedis, Gelidiales, Gigartinales, Gracilariales, Halymeniales, Hildenbrandiales, Nemaliales, Nemastomatales, Palmariales, Peyssonneliales, Pihiellales, Plocamiales, Rhodachlyales, Rhodogorgonales, Rhodymeniales, Sebdeniales, Sporolithales and Thoreales.

Red algae contain pigments encountered in the other plants, such as chlorophyll and carotenoids, but their originality lies in the presence of phycobiliproteins: allophycocyanin (blue), phycocyanin (blue) and phycoerythrin which gives the red color. The chloroplast organization differentiates red algae from glaucophytes and cyanobacteria.

The pigmentation of the red alga depends in part on the wavelength of the light which reaches the alga. At depth, red algae accumulate a large amount of phycoerythrin, which is a pigment that can absorb light at this depth. At the surface, the ratio of the red pigment decreases, relative to the chlorophyll and to the other phycobiliproteins; they become greener despite their name; this is referred to as chromatic adaptation.

Red algae, in particular of the Gelidium genus and of the Palmaria genus, are highly used in cosmetics, for various applications. Korean patent application published under number KR 101409764 describes the use of an extract of Gelidium amansii, obtained from a production process by lactic fermentation, as antiwrinkle ingredients, for the cosmetics and nutrition industries.

The international application published under number WO 2013/178965 A2 describes the use of algal extracts, in particular extracts of red algae such as Pterocladia capillacea and Palmaria palmata, as mitophagy-activating ingredients, resulting in a detoxifying effect.

The French patent application published under number FR 2 911 278 A1 describes the use of an extract of the red alga Palmaria palmata, obtained by carrying out an enzymatic process, followed by steps of filtration, of decanting, of centrifugation, then of deactivation of the residual enzymes by heating, of decoloring and of concentration by carrying out techniques known to those skilled in the art, in order to obtain a sugar-rich extract having human skin-depigmenting properties, intended for the cosmetics industry.

The Korean patent published under number KR 10 112 8591 B1 describes the use of extracts of the red algae Porphyra tenera and Gelidium amansii in a cosmetic composition for topical use, as an agent intended for protecting human skin from the harmful and unattractive effects of ultraviolet radiation.

Mention may thus be made of the extract of the brown alga Ascophyllum nodosum, sold by the company Algues & Mer under the trade name Invicity™, described as reinforcing the barrier function of human skin, by decreasing the activity of polluting agents of hydrocarbon type (AhR). Mention may also be made of the extract of the red alga Asparagopsis armata, sold by the company Algues & Mer under the trade name Ysaline™, described as making it possible to eliminate part of the bacterial and fungal flora of the skin.

Among the ten thousand different species of macroalgae, only about a hundred of them are at the current time of economic interest. Thus, certain food macroalgae, hydrocolloid-producing algae and algae producing molecules with biological activity have been the subject of extensive research in order to better understand their physiology, their metabolism and their reproduction.

The algae used in food for human consumption, such as Undaria pinnatifida, more well known as Wakame, Laminaria japonica, also known as Kombu, Porphyra yezoensis, also known as Nori and Enteromorpha intestinalis, also known as Ao Nori, have been studied in order to master the culture thereof.

The hydrocolloids produced by algae such as Chondrus crispus, Kappaphycus spp. and Eucheuma denticulatum are mainly used as thickeners and gelling agents in food and cosmetic products. The biomasses producing these polymers are of definite economic interest [Ronelie C. et al. in “Non-enzymatic isolation of somatic cells from Kappaphycus spp. and Eucheuma denticulatum (Solieriaceae, Rhodophyta)”; Eur. J. Phycol. (2014), 49(4): 486-492]. In this regard, Clinton J. Dawes et al discloses a process for obtaining a unialgal biomass of cells of the red algae Eucheuma denticulatum and Kappaphycus alvarezfi, from a sample taken from the natural environment, and the culture thereof for harvesting the biomass [Clinton J. Dawes et al in: “Branch, micropropagule and tissue culture of the red algae Eucheuma denticulatum and Kappaphycus alvarezii farmed in the Philippines”, Journal of applied Phycology 3: 247-257, 1991]. The same is true for Myounghoon et al. in the case of the red alga Galdieria sulphuraria [Myounghoon et al. in: “Isolation and characterization of thermostable phycocyanin from Galdieria suphuraria”, Korean J. Chem. Eng., 31(3), 490-495].

The bioreactor culture of the green alga Acrosiphonia coalita, the brown alga Laminaria saccharina and the red algae Agardhiella subulata, Ochtodes secundiramea and Portieria hornemannii has been studied with the aim of synthesizing molecules with biological activity [Gregory L. Rorrer et al. Production of bioactive metabolites by cell and tissue cultures of marine macroalgae in bioreactor systems. Plant Cell and Tissue Culture for the Production of Food Ingredients, edited by Fu et al. Kluwer Academic/Plenum Publishers, New York, 1999].

Some of them are based on the knowledge and the control of the reproductive cycle of the acroalga. For example, in laminarials, it is possible to observe the alternation between a macroscopic diploid sporophyte developed in thallus and microscopic male gametophytes and haploid females. The fertile mature sporophytes produce swimming spores which are deposited on a solid substrate and which give rise to gametophytes. The study of their life cycle, such as that of Undaria pinnatifida, more well known by the name Wakame, or of Laminaria japonica, known as Kombu, has made it possible to develop the culture of gametophytes, a microscopic life form of these algae for inoculating supports on which will grow macroscopic sporophytes which can be exploited in food for human consumption. The culture of these algae is carried out first in the laboratory for culturing the microscopic life form and then in the natural environment for producing the macroscopic life form which it will be possible to consume. The development of the culture of the sporophyte of brown algae of the order Laminariales, such as Undaria pinnatifida, in the 1980s led to the development of a gametophyte culture technique termed “free living culture”, which consists in harvesting mature sporophytes, bringing about sporulation, capturing the spores, forming gametophytes and then culturing them for the purpose of creating gametes which, after fertilization, will give rise to new sporophytes (R. Perez et al., 1984).

The development of the culture of the Laminaria saccharina sporophyte also required the development of a method of culture of its gametophyte, in a manner similar to the cultures of Undaria pinnatifida [C. Zhi, G. L. Rorrer. Photolithotrophic cultivation of Laminaria saccharina gametophyte cells in a bubble-column bioreactor [Enzyme and Microbial Technology. Volume 18, Issue 4, March 1996, Pages 291-299]].

Likewise, for the purposes of experimental experiments, H. Stegenga et al. isolated, by culturing samples of Chromastrum moniliforme, gametophytes and tetrasporophytes of these algae [H. Stegenga et al. in: “Remarks on the Audouinella microscopica (NÄG.) Woekerling complex, with a brief survey of the genus chromastrum papenfuss (Rhodophyta, Nemaliales)”, Acta Bot. Neerl. 28(4/5), August 1979, p. 289-311].

The Chinese patent application published under number CN 103858745 A discloses the development of artificial cultures of Sytosiphon lomentaria by controlling the steps of differentiation of the germinated plasma of the alga, for producing a mature unilocular sporange, then bringing about the sporulation thereof with the aim of inoculating culture supports for the purpose of producing, in the sea, macroscopic Sytosiphon lomentaria biomass.

The Chinese patent application published under number CN 103931482 A discloses a method for obtaining the thallus of the gametophyte of the red alga Porphyra yezoensis, which is used in food for human consumption, in particular for the production of maki, involving first the “in vitro” culture of the conchocelis phase of the alga in order to produce concho spores which will bind to a culture support to enable thallus growth in the sea.

Other methods involve the induction of calluses from red algal explants, said calluses leading to the development of plantlets which are then cultured in a bioreactor. In this context, mention may be made of the studies of Ronelie et al. [Ronelie C. et al. Nonenzymatic isolation of somatic cells from Kappaphycus spp. and Eucheuma denticulatum (Solieriaceae, Rhodophyta), Eur. J. Phycol. (2014), 49(4): 486-492]; those of J. Munoz [J. Munoz. Use of plant growth regulators in micropropagation of Kappaphycus alvarezii (Doty) in airlift bioreactors. J Appl Phycol (2006) 18:209-218], or those of Maliakal et al. [S. Maliakal, D. Cheney. Halogenated monoterpenes production in regenerated plantlet cultures of Ochtodes secundiramea. J. Phycol. 37,1010-1019 (2001)].

Other methods involve the production of protoplasts from a thallus with the aim of re-forming new thalluses, using, as proposed by Rusig et al. in the case of Enteromorpha intestinalis, which is an alga used in food for human consumption and animal feed, an enzymatic mixture containing cellulase and Aplysia enzymes, which makes it possible to digest the wall of the algal thallus cells. The cells of which the wall is digested are called protoplasts [A. Rusig & J. Cosson, Plant regeneration from protoplasts of Enteromorpha intestinalis (Chlorophyta, Ulvophyceae) as seedstock for macroalgal culture. Journal of Applied Phycology 13: 103-108, 2001].

As small multicellular algae are not sufficiently abundant in nature to allow harvesting from the environment and since their small size makes it difficult to identify the desired species and also to specifically harvest a given species, a process may be carried out which makes it possible to obtain a unialgal biomass of small multicellular macroalga cells, in order to be able to extract therefrom an active ingredient that can be used in the cosmetics industry.

This process has the advantage of being usable in a bioreactor, and of not requiring the use of axenic explants, and does not involve phytohormones or enzymes.

In the context of their research into new cosmetic active ingredients for the prevention and/or treatment of the unattractive signs generated by the polluting agents present in the atmosphere, on the skin, the scalp, the hair or the mucous membranes, the inventors have endeavored to develop a new technical solution consisting of a process of which the aim is to prevent or slow the appearance of said unattractive signs or else to eliminate them, using glycolic extracts (GEs) of a unialgal biomass of small multicellular macroalga cells originating from the class Florideophyceae.

For this reason, according to a first aspect, a subject of the invention is a process for preventing or slowing the appearance, on the skin, the scalp, the hair or the mucous membranes, of unattractive signs generated by the polluting agents present in the atmosphere or else for eliminating said signs, said process comprising at least one step of applying, to human skin, to the mucous membranes, to the scalp or to the hair, a cosmetic formulation for topical use comprising at least one cosmetically acceptable excipient and an effective amount of a glycolic extract (GE) of a unialgal biomass of small multicellular macroalga cells originating from the class Florideophyceae, said extract being obtained by the process comprising the following successive steps:
a step A) of preparing a unialgal sample of multicellular macroalga cells from a sample of macroalgae originating from the class Florideophyceae and taken from the natural environment;
a step B) of culturing said unialgal sample of multicellular macroalga cells obtained in step A) in seawater supplemented with at least one nitrogen source, so as to obtain an aqueous suspension of said unialgal biomass of small multicellular macroalga cells;
a step C) of harvesting said unialgal biomass of small multicellular macroalga cells from said aqueous suspension obtained at the end of step B);
optionally, a step D) of preparing a powder of said unialgal biomass of small multicellular macroalga cells obtained in step C);
a step E) during which said unialgal biomass of small multicellular macroalga cells obtained in step C) or the powder of said biomass in step D) is dispersed with stirring in a water-glycol mixture in a proportion of from 1% by weight to 20% by weight of biomass per 100% by weight of dispersion; and
a step F) during which the dispersion obtained in the preceding step E) is separated into its immiscible phases, so as to collect said glycolic extract (GE).

In the cosmetic process as described above, the cosmetic formulation for topical use is spread over the surface of the skin, or of the scalp, or of the mucous membranes, or of the hair to be treated, then said surface is massaged for a few moments.

The expression “for topical use” used in the definition of the cosmetic formulation used in the cosmetic process which is a subject of the present invention means that said formulation is used by application to the skin, whether it is a direct application in the case of a cosmetic formulation, or an indirect application when the cosmetic formulation according to the invention is impregnated onto a support intended to be brought into contact with the skin (paper, wipe, textile, transdermal device, etc.).

The expression “cosmetically acceptable” used in the definition of the cosmetic formulation for topical use, used in the cosmetic process which is a subject of the present invention, means, according to the Council of the European Economic Community Directive no. 76/768/EEC of Jul. 27, 1976, amended by Directive no. 93/35/EEC of Jun. 14, 1993, that said formulation comprises any substance or preparation intended to be brought into contact with the various parts of the human body (epidermis, body hair and head hair system, nails, lips and genitals) or with the teeth and mucous membranes of the mouth, for the purpose, exclusively and mainly, of cleansing them, fragrancing them, modifying the appearance thereof and/or correcting body odors thereof and/or protecting them or keeping them in good condition.

The term “effective amount” of the glycolic extract (GE) as defined previously and present in the cosmetic formulation for topical use used in the process as defined above is intended to mean, for 100% of the weight of said cosmetic formulation for topical use, the amount of between 0.1% and 5% by weight, more particularly between 0.1% and 3% by weight, and even more particularly between 0.5% and 2% by weight of the glycolic extract (GE).

For the purposes of the present invention, the term “small multicellular macroalga” denotes a multicellular macroalga between 30 μm and 3 cm in size and organized in cell clumps. This small multicellular macroalga differentiates from a multicellular macroalga which is large since the latter is between 5 cm and 20 cm in size and is organized into a tissue.

Particularly denoted as sample of a macroalga taken from the natural environment, used in step A) of the process for preparing the glycolic extract (GE) as defined above, is a sample taken from seawater, whether it is a sample of seawater taken, a sample taken at the surface of solid substrates such as rocks, sand, shells, sediments or else artificial supports such as a boat hull, a pontoon or a sea wall; it may also be a sample taken at the surface of or from inside other plants (epiphyte or endophyte), such as marine plants or algae, at the surface of or from inside animals (epiphyte or endophyte), such as sponges, cnidarians, protochordates, echinoderms, molluscs, arthropods, annelids, or marine vertebrates.

The macroalgal sample taken from the natural environment, used in step A) of the process for preparing the glycolic extract (GE) as defined above, is generally very rich in biodiversity and contains a vast selection of living organisms, such as small animals, protozoa, prokaryotic microalgae, eukaryotic microalgae and multicellular macroalgae.

The expression “unialgal sample of multicellular macroalga cells” is intended to mean, in the process as defined above, a culture containing just one species of multicellular algae.

According to step A) of the process for preparing the glycolic extract (GE) as defined above, the unialgal sample of small multicellular macroalga cells is obtained by isolating the targeted macroalgae from the other organisms of the targeted alga. To this effect, physical separation means and/or chemical separation means may be used.

As physical separation means, there is, for example, separation performed by means of a glass pipette using the end of the capillary tube to cut away a few targeted small multicellular macroalga cells, while visually controlling the operation under a microscope or under a binocular magnifying lens. There is also separation by successive dilution of the cells of the targeted species from the natural sample.

As chemical separation means, there is, for example, the use of antibiotics for eliminating microalgae of the cyanobacteria type, or the use of germanium dioxide for eliminating microalgae of the diatomaceous type.

The various isolation means are combined in order to obtain the best possible isolation result. All the physical and chemical isolation methods are carried out in translucent receptacles which allow light to pass through, containing sterile seawater, containing at least one source of nitrogen such as sodium nitrate (NaNO₃) at a concentration of between 50 mg/dm³ and 250 mg/dm³ with a preference for 150 mg/dm³, and a source of phosphorus such as sodium dihydrogen phosphate (NaH₂PO₄) at a concentration of between 5 mg/dm³ and 75 mg/dm³ with a preference for 50 mg/dm³. It is also possible to add other mineral elements to the seawater, by addition for example of a nutritive medium in the desired proportions, such as Provasoli's medium having the following composition:

Provasoli's Medium

| | | | | | |
|---|---|---|---|---|---|
| NaNO₃ | 350 | mg | ZnSO₄•7H₂O | 0.55 | mg |
| sodium glycerophosphate | 50 | mg | CoSO₄•7H₂O | 0.12 | mg |
| Fe(NH₄)₂(SO₄)₂•6H₂O | 18 | mg | Vitamin B12 | 10 | μg |
| Na₂EDTA | 15 | mg | Thiamine | 0.5 | mg |
| H₃BO₃ | 28.5 | mg | Biotin | 5 | μg |
| FeCl₃•6H₂O | 1.225 | mg | Tris Buffer | 500 | mg |
| MnSO₄•H₂O | 4.1 | mg | distilled water | 100 | ml |

The implementation of step B) of the process for preparing the glycolic extract (GE) as defined above is carried out in a photobioreactor containing seawater containing at least one source of nitrogen such as sodium nitrate (NaNO₃) at a concentration of between 50 mg/dm³ and 250 mg/dm³ with a preference for 150 mg/dm³, and a source of phosphorus such as sodium dihydrogen phosphate (NaH₂PO₄) at a concentration of between 5 mg/dm³ and 75 mg/dm³ with a preference for 50 mg/dm³.

It is also possible to add other mineral elements to the seawater, by addition for example of Provasoli's medium in the desired proportions. The cultures are carried out in translucent culture tanks with bubbling of air optionally enriched with carbon dioxide.

The cultures are generally carried out at between 10° C. and 25° C. with a preference for 17° C., under constant illumination.

The cultures are carried out over periods of fifteen days in volumes ranging from 500 cm³, for the first steps of the culture, up to 20 m³ for the industrial biomass production steps. However, when observations under a binocular magnifying lens and/or under a microscope carried out at the end of the first fifteen-day period demonstrate the growth of algae other than the one targeted at the end of step A) of the process for preparing the glycolic extract (GE), this constitutes the sign that the result of said step A) is not satisfactory. This step A) of the process for preparing the glycolic extract (GE) as defined previously is therefore repeated until a unialgal sample of small multicellular macroalga cells of satisfactory quality is obtained.

Step C) of the process for preparing the glycolic extract (GE) as defined above is generally carried out using a filtering cloth with a cut-off threshold of between 25 μm and 100 μm as a function of the size of the small multicellular macroalgae cultured. The culture of unialgal biomass of small multicellular macroalga cells is filtered, the seawater passing through the cloth and the biomass remaining at its surface. The retentate constituted by the biomass is then pressed so as to remove the free water still present.

Step D) of the process for preparing the glycolic extract (GE) as defined above is carried out by methods known to those skilled in the art. It is possible for example to dry said unialgal biomass of small multicellular macroalga cells, obtained in step C) of the process for preparing the glycolic extract (GE) as defined above, then to mill it and to sieve it so as to obtain a powder of desired average diameter. According to one particular aspect of the process which is the subject of the present invention, during step D) of the process for preparing the glycolic extract (GE) as defined above, said unialgal biomass of small multicellular macroalga cells, obtained in step C), is frozen, freeze-dried and then milled so as to obtain the desired powder.

The water-to-glycol volume ratio of the water/glycol mixture used in the process for preparing the glycolic extract (GE) as defined above is generally less than or equal to 1/1 and greater than or equal to 1/9 and more particularly less than or equal to 1/2 and greater than or equal to 1/6. If necessary or if desired, this volume ratio is adjusted within the abovementioned range at the end of step E) of the process for preparing the glycolic extract (GE).

During step F) of the process for preparing the glycolic extract (GE) as defined above, the separation of the immiscible phases is carried out by decanting, by gravity or by centrifugation. If necessary or if desired, the aqueous-glycolic extract obtained is filtered through a filter with a cut-off threshold of 0.2 μm in order to remove any particle in suspension and to make it possible to obtain a clear solution.

As glycol used in step E) of the process for preparing the glycolic extract (GE) as defined above, mention may in particular be made of 1,4-butanediol, 1,3-butanediol, 1,2-butanediol, 1,2-pentanediol, 1,5-pentanediol, 1,2-hexanediol or 1,6-hexanediol. According to one particular aspect of the process which is the subject of the present invention, the glycol used in step E) of the process for preparing the glycolic extract (GE) as defined above is 1,3-butanediol.

For the purposes of the present invention, the expression “unattractive signs generated by the polluting agents present in the atmosphere, on the skin, the scalp, the hair or the mucous membranes” is intended to mean any modification of the external appearance of the skin, the scalp, the hair or the mucous membranes due to the consequences of exposure to atmospheric polluting agents, such as redness, wrinkles and fine lines, degradation of the microrelief of the skin, of the scalp or of the mucous membranes, dulling of the skin complexion, of the scalp and of the mucous membranes, or an increase in the porosity, dulling and weakening of the hair with respect to mechanical stresses.

For the purposes of the present invention, the term “polluting agents present in the atmosphere” is intended to mean toxic gases, for instance carbon monoxide, nitrogen oxides such as nitrogen dioxide, sulfur oxides such as sulfur dioxide, ozone; metals, for instance cobalt, mercury, cadmium, iron and nickel; atmospheric particles which are in the form of suspensions in the Earth's atmosphere, for instance the microparticles identified under the acronyms PM2.5 and PM10, cigarette smoke.

According to a more particular aspect of the present invention, a subject thereof is the process as defined above for which step E) of the process for preparing the glycolic extract (GE), as defined above, is characterized by the dispersion of said unialgal biomass of small multicellular macroalga cells, obtained in step C) or in step D), the water/glycol mixture used is a water/1,3-butanediol mixture, the weight content of 1,3-butanediol of which (weight ratio of 1,3-butanediol to total weight of 1,3-butanediol and water) is between 50% and 75%, in a proportion of 2% by weight to 10% by weight of biomass per 100% by weight of dispersion; and for which the stirring is maintained for 1 to 2 hours, then, if necessary or if desired, the weight content of 1,3-butanediol is adjusted to 40% by adding water.

According to another particular aspect of the present invention, a subject thereof is the process as defined above, for which said small multicellular macroalga cells originating from the class Florideophyceae, constituting said unialgal biomass, originate from the subclass Nemaliophycidae, order Acrochaetiales, family Acrochatiaceae, genus Acrochaetium and species Acrochaetium moniliforme.

According to this particular aspect of the process which is a subject of the present invention, the powder of said unialgal biomass of small multicellular macroalga cells used in step E) of the process for preparing the glycolic extract (GE) as defined above is a powder of said unialgal biomass of cells of red algae originating from the class Florideophyceae, in the subclass Nemaliophycidae, order Acrochaetiales, family Acrochatiaceae, genus Acrochaetium and species Acrochaetium moniliforme.

The cosmetic formulations for topical use that are used in the process having the aim of preventing or slowing the appearance of the unattractive signs generated by the polluting agents present in the atmosphere, on the skin, the scalp, the hair or the mucous membranes, or else of eliminating said signs, which are a subject of the present invention and as defined above, are generally in the form of an aqueous or aqueous-alcoholic or aqueous-glycolic solution, in the form of a suspension, an emulsion, a microemulsion or a nanoemulsion, regardless of the water-in-oil, oil-in-water, water-in-oil-in-water or oil-in-water-in-oil type, or in the form of a powder.

The cosmetic formulations for topical use that are used in the process having the aim of preventing or slowing the appearance of the unattractive signs generated by the polluting agents present in the atmosphere, on the skin, the scalp, the hair or the mucous membranes, or else of eliminating said signs, which are a subject of the present invention and as defined above, can be packaged in a bottle, in a device of pump-dispenser “bottle” type, in pressurized form in an aerosol device, in a device which has a perforated wall such as a grid, or in a device which has a roll-on applicator.

In general, the glycolic extract (GE), obtained according to the preparation process as defined above, and present in the cosmetic formulations for topical use that are used in the process which is a subject of the present invention, is combined with chemical additives normally used in the field of formulations for topical use, such as foaming and/or detergent surfactants, thickening and/or gelling surfactants, thickeners and/or gelling agents, anti-pollution agents, stabilizers, film-forming compounds, solvents and cosolvents, hydrotropic agents, spring or mineral waters, plasticizers, emulsifiers and co-emulsifiers, opacifiers, pearlescent agents, superfatting agents, sequestering agents, chelating agents, oils, waxes, antioxidants, fragrances, essential oils, preservatives, conditioning agents, deodorants, whitening agents intended for bleaching body hair and the skin, active ingredients intended to provide a treating and/or protective action with respect to the skin or the hair, sunscreens, mineral fillers or pigments, particles which provide a visual effect or are intended for encapsulating active agents, exfoliating particles, texturing agents, optical brighteners, insect repellents.

As “anti-pollution agents” that can be combined with the glycolic extract (GE), obtained according to the preparation process as defined above, and present in the cosmetic formulations, for topical use that are used in the process which is a subject of the present invention, mention may be made of compounds capable of trapping ozone, compounds capable of trapping monocyclic or polycyclic aromatic compounds such as benzopyrene, metals and heavy metals.

Among the ozone-trapping agents that can be combined with the glycolic extract (GE), obtained according to the preparation process as defined above, and present in the cosmetic formulations for topical use that are used in the process which is a subject of the present invention, mention may be made of phenols and polyphenols, in particular tannins; olive leaf extracts; extracts of tea, in particular of green tea; anthocyanins; extracts of rosemary; phenolic acids, in particular chlorogenic acid; stilbenes, in particular resveratrol; derivatives of sulfur-containing amino acids, in particular S-carboxymethylcysteine; ergothioneine; N-acetylcysteine; chelating agents such as N,N′-bis(3,4,5-trimethoxybenzyl)ethylenediamine or a salt, metal complex or ester thereof; carotenoids such as crocetin; and various starting materials such as the mixture of arginine, ribonucleate, histidine, mannitol, adenosinetriphosphate, pyridoxine, phenylalanine, tyrosine and hydrolyzed RNA sold by Laboratoires Serobiologiques under the trade name CPP LS 2633-12F™, the water-soluble fraction of corn sold by the company Solabia under the trade name Phytovityl™, the mixture of fumitory extract and lemon extract sold under the name Unicotrozon C-490 by the company Induchem, and the mixture of ginseng, apple, peach, wheat and barley extracts, sold by the company Provital under the trade name Pronalen Bioprotect™.

Among the agents trapping monocyclic or polycyclic aromatic compounds that can be combined with the glycolic extract (GE), obtained according to the preparation process as defined above, and present in the cosmetic formulations for topical use that are used in the process which is a subject of the present invention, mention may be made of indole derivatives, in particular indole-3-carbinol; extracts of tea, in particular of green tea, extracts of water hyacinth or Eichornia crassipes; and the water-soluble fraction of corn sold by the company Solabia under the trade name Phytovityl™.

Among the heavy-metal-trapping agents that can be combined with the glycolic extract (GE), obtained according to the preparation process as defined above, and present in the cosmetic formulations for topical use that are used in the process which is a subject of the present invention, mention may be made of chelating agents such as EDTA, the pentasodium salt of ethylenediaminetetramethylenephosphonic acid, and N,N′-bis(3,4,5-trimethoxybenzyl)ethylenediamine or a salt, metal complex or ester thereof; phytic acid; chitosan derivatives; extracts of tea, in particular of green tea; extracts of water hyacinth (Eichornia crassipes); and the water-soluble fraction of corn sold by the company Solabia under the trade name Phytovityl™.

Among the solvents, and more particularly among the volatile solvents, that can be combined with the glycolic extract (GE), obtained according to the preparation process as defined above, and present in the cosmetic formulations for topical use that are used in the process which is a subject of the present invention, mention may be made of water-soluble and volatile alcohols, such as ethanol, isopropanol or butanol, and more particularly ethanol, organic solvents such as glycerol, diglycerol, glycerol oligomers, propane-1,2-diol, propane-butane-1,3-diol, butane-1,4-diol, hexane-1,6-diol, 2-methylpentane-2,4-diol, pentane-1,2 diol, 2-methylpentane-2,4-diol (or hexylene glycol), dipropylene glycol, xylitol, erythritol or sorbitol.

Among the foaming and/or detergent surfactants that can be combined with the glycolic extract (GE), obtained according to the preparation process as defined above, and present in the cosmetic formulations for topical use that are used in the process which is a subject of the present invention, mention may be made of anionic, cationic, amphoteric or nonionic foaming and/or detergent surfactants.

Among the foaming and/or detergent anionic surfactants that may be combined with the glycolic extract (GE), obtained according to the preparation process as defined above, and present in the cosmetic formulations for topical use that are used in the process which is a subject of the present invention, mention may be made of alkali metal salts, alkaline-earth metal salts, ammonium salts, amine salts, amino alcohol salts of alkyl ether sulfates, of alkyl sulfates, of alkylamido ether sulfates, of alkylarylpolyether sulfates, of monoglyceride sulfates, of a-olefin sulfonates, of paraffin sulfonates, of alkyl phosphates, of alkyl ether phosphates, of alkyl sulfonates, of alkylamide sulfonates, of alkylaryl sulfonates, of alkyl carboxylates, of alkylsulfosuccinates, of alkyl ether sulfosuccinates, of alkylamide sulfosuccinates, of alkyl sulfoacetates, of alkyl sarcosinates, of acylisethionates, of N-acyl taurates, of acyl lactylates, of N-acylamino acid derivatives, of N-acyl peptide derivatives, of N-acyl protein derivatives or of fatty acids.

Among the amphoteric foaming and/or detergent surfactants that can be combined with the glycolic extract (GE), obtained according to the preparation process as defined above, and present in the cosmetic formulations for topical use that are used in the process which is a subject of the present invention, mention may be made of alkylbetaines, alkylamidobetaines, sultaines, alkylamidoalkylsulfobetaines, imidazoline derivatives, phosphobetaines, amphopolyacetates and amphopropionates.

Among the cationic foaming and/or detergent surfactants that can be combined with the glycolic extract (GE), obtained according to the preparation process as defined above, and present in the cosmetic formulations for topical use that are used in the process which is a subject of the present invention, mention may be made particularly of quaternary ammonium derivatives.

Among the foaming and/or detergent nonionic surfactants that can be combined with the glycolic extract (GE), obtained according to the preparation process as defined above, and present in the cosmetic formulations for topical use that are used in the process which is a subject of the present invention, mention may be made more particularly of alkylpolyglycosides comprising a linear or branched, saturated or unsaturated aliphatic radical, and comprising from 8 to 12 carbon atoms, such as for example octylpolyglucoside, decylpolyglucoside, laurylpolyglucoside, cocoylpolyglucoside, or mixtures thereof; castor oil derivatives, polysorbates, copra amides or N-alkylamines.

As examples of texturing agents that can be combined with the glycolic extract (GE), obtained according to the preparation process as defined above, and present in the cosmetic formulations for topical use that are used in the process which is a subject of the present invention, mention may be made of N-acylated derivatives of amino acids, for example lauroyl lysine sold under the name Aminohope™LL, octenyl starch succinate sold under the name Dryflo™, myristyl polyglucoside sold under the name Montanov™ 14, cellulose fibers, cotton fibers, chitosan fibers, talc, sericite, mica or perlite.

As examples of active ingredients that can be combined with the glycolic extract (GE), obtained according to the preparation process as defined above, and present in the cosmetic formulations for topical use that are used in the process which is a subject of the present invention, mention may be made of:
vitamins and derivatives thereof, for example retinol (vitamin A) and esters thereof (for example retinyl palmitate), ascorbic acid (vitamin C) in the form of the salts and esters thereof, sugar derivatives of ascorbic acid (for example ascorbyl glucoside), tocopherol (vitamin E) and esters thereof (for example tocopheryl acetate), vitamins B3 or B10 (niacinamide and derivatives thereof);
compounds with a lightening or depigmenting action on the skin, for example Sepiwhite™ MSH, arbutin, kojic acid, hydroquinone, Vegewhite™, Gatuline™, Synerlight™ Biowhite™, Phytolight™, Dermalight™, Clariskin™, Melaslow™, Dermawhite™, Ethioline, Melarest™, Gigawhite™, Albatine™, Lumiskin™, Seashine™, Celtosome™ Crithmum maritimum;
compounds with a calmative action, such as Sepicalm™ S, allantoin and bisabolol;
anti-inflammatory agents;
compounds with moisturizing action, for example diglycerol, triglycerol, urea, hydroxyureas, glycerol glucoside, diglycerol glucoside, polyglyceryl glucosides, erythrityl glucoside, sorbityl glucoside, xylityl glucoside, the composition sold under the brand name Aquaxyl™ comprising xylityl glucoside, anhydroxylitol and xylitol; the algal extracts sold under the brand names Codiavelane™ and Bioplasma™;
compounds with slimming or lipolytic action, such as caffeine or derivatives thereof, Adiposlim™, Adipoless™, Rodysterol™;
N-acyl proteins; N-acyl peptides, for example Matrixil™; N-acyl amino acids; partial hydrolyzates of N-acyl proteins; amino acids; peptides; total protein hydrolyzates;
plant extracts rich in tannins, polyphenols and/or isoflavones, for example grape extracts, pine extracts, wine extracts, olive extracts; soybean extracts, for example Raffermine™; wheat extracts, for example Tensine™ or Gliadine™; terpene-rich plant extracts; freshwater or seawater algal extracts; marine extracts in general such as corals;
compounds with antimicrobial action or with purifying action, for example Lipacide™ CBG, Lipacide™ UG, Sepicontrol™ A5; Octopirox™ or Sensiva™ SC50, Phlorogine™;
compounds with an energizing or stimulating property, such as Physiogenyl™, panthenol and derivatives thereof such as Sepicap™ MP, Sepitonic™ M3, Sepitonic™ M4;
anti-aging active agents, such as Sepilift™ DPHP, Lipacide™ PVB, Sepivinol™, Sepivital™, Manoliva™, Phyto-Age™, Timecode™; Survicode™, Kalpariane™, Antileukine-6™, Ephemer™, Juvenessence™, Celtosomes™ Erygium maritimum, Ceramoside™;
active agents for combating photoaging;
active agents for increasing the synthesis of extracellular matrix components, for example collagen, elastins and glycosaminoglycans;
active agents acting favorably on chemical cellular communication, such as cytokines, or physical cellular communication, such as integrins;
active agents which create a “heating” sensation on the skin, such as skin capillary circulation activators (for example nicotinic acid derivatives) or products which create a “freshness” sensation on the skin (for example menthol and derivatives thereof);
active agents which improve the skin capillary circulation, for example venotonic agents; draining active agents; decongesting active agents, for example extracts of Ginkgo biloba, ivy, common horse chestnut, bamboo, ruscus, butcher's-broom, Centella asiatica, fucus, rosemary or willow;
active agents acting as skin-tautening agents, for example plant protein hydrolyzates, hydrolyzates of marine origin, for instance hydrolyzates of laminaria extracts, fish cartilage hydrolyzates, marine elastin, the product sold by the company SEPPIC under the brand name Sesaflash™, and collagen solutions;
agents for tanning or browning the skin, for example dihydroxyacetone, isatin, alloxan, ninhydrin, glyceraldehyde, mesotartaric aldehyde, glutaraldehyde, erythrulose, plant extracts, for example extracts of redwoods of the genus Pterocarpus and of the genus Baphia, such as Pteropcarpus santalinus, Pterocarpus osun, Pterocarpus soyauxii, Pterocarpus erinaceus, Pterocarpus indicus or Baphia nitida, such as those described in European patent application EP 0 971 683; agents known for their action in facilitating and/or accelerating tanning and/or browning of human skin, and/or for their action in coloring human skin, for example carotenoids (and more particularly beta-carotene and gamma-carotene), the product sold under the brand name “Carros oil” (INCI name: Daucus carota, Helianthus annuus Sunflower oil) by the company Provital, which contains carotenoids, vitamin E and vitamin K; tyrosine and/or the derivatives thereof, known for their effect on accelerating tanning of the human skin in combination with exposure to ultraviolet radiation, for example the product sold under the trade name “SunTan Accelerator™” by the company Provital, which contains tyrosine and riboflavins (vitamin B), the tyrosine and tyrosinase complex sold under the trade name “Zymo Tan Complex” by the company Zymo Line, the product sold under the trade name MelanoBronze™ (INCI name: Acetyl Tyrosine, Monk's pepper extract (Vitex Agnus-castus) by the company Mibelle, which contains acetyl tyrosine, the product sold under the trade name Unipertan VEG-24/242/2002 (INCI name: butylene glycol and Acetyl Tyrosine and hydrolyzed vegetable protein and Adenosine triphosphate) by the company Unipex, the product sold under the trade name “Try-Excell™” (INCI name: Oleoyl Tyrosine and Luffa Cylindrica (Seed Oil and Oleic acid) by the company Sederma, which contains extracts of marrow seed (or loofah oil), the product sold under the trade name Actibronze™ (INCI name: hydrolyzed wheat protein and acetyl tyrosine and copper gluconate) by the company Alban Muller, the product sold under the trade name Tyrostan™ (INCI name: potassium caproyl tyrosine) by the company Synerga, the product sold under the trade name Tyrosinol (INCI name: Sorbitan Isostearate, glyceryl oleate, caproyl Tyrosine) by the company Synerga, the product sold under the trade name InstaBronze™ (INCI name: Dihydroxyacetone and acetyl tyrosine and copper gluconate) sold by the company Alban Muller, the product sold under the trade name Tyrosilane (INCI name: methylsilanol and acetyl tyrosine) by the company Exymol; peptides known for their melanogenesis-activating effect, for example the product sold under the trade name Bronzing SF Peptide powder (INCI name: Dextran and Octapeptide-5) by Infinitec Activos, the product sold under the trade name Melitane (INCI name: Glycerin and Aqua and Dextran and Acetyl hexapeptide-1) comprising the acetyl hexapeptide-1 known for its alpha-MSH agonist action, the product sold under the trade name Melatimes Solutions™ (INCI name: Butylene glycol, Palmitoyl Tripeptide-40) by the company Lipotec, sugars and sugar derivatives, for example the product sold under the trade name Tanositol™ (INCI name: inositol) by the company Provital, the product sold under the trade name Thalitan™ (or Phycosaccharide™ AG) by the company CODIF international (INCI name: Aqua and hydrolyzed algin (Laminaria Digitata) and magnesium sulfate and manganese sulfate) containing an oligosaccharide of marine origin (guluronic acid and mannuronic acid chelated with magnesium and manganese ions), the product sold under the trade name Melactiva™ (INCI name: Maltodextrin, Mucuna Pruriens Seed extract) by the company Alban Muller, flavonoid-rich compounds, for example the product sold under the trade name “Biotanning” (INCI name: Hydrolyzed citrus Aurantium dulcis fruit extract) by the company Silab and known to be rich in lemon flavonoids (of the hesperidin type); agents intended for treating head hair and/or body hair, for example agents for protecting the melanocytes of the hair follicle, intended to protect said melanocytes against cytotoxic agents responsible for the senescence and/or apoptosis of said melanocytes, such as mimetics of DOPAchrome tautomerase activity, selected from those described in the European patent application published under the number EP 1 515 688 A2, the synthetic SOD mimetic molecules, for example manganese complexes, antioxidant compounds, for example cyclodextrin derivatives, silica-containing compounds derived from ascorbic acid, lysine or arginine pyrrolidone carboxylate, combinations of mono- and diesters of cinnamic acid and of vitamin C, and more generally those mentioned in the European patent application published under the number EP 1 515 688 A2.

As examples of deodorants that can be combined with the glycolic extract (GE), obtained according to the preparation process as defined above, and present in the cosmetic formulations for topical use that are used in the process which is a subject of the present invention, mention may be made of alkali metal silicates, zinc salts such as zinc sulfate, zinc gluconate, zinc chloride or zinc lactate; quaternary ammonium salts such as cetyltrimethylammonium salts or cetylpyridinium salts; glycerol derivatives such as glyceryl caprate, glyceryl caprylate and polyglyceryl caprate; 1,2-decanediol; 1,3-propanediol; salicylic acid; sodium bicarbonate; cyclodextrins; metallic zeolites; Triclosan™; aluminum bromohydrate, aluminum chlorohydrates, aluminum chloride, aluminum sulfate, aluminum zirconium chlorohydrates, aluminum zirconium trichlorohydrate, aluminum zirconium tetrachlorohydrate, aluminum zirconium pentachlorohydrate, aluminum zirconium octachlorohydrate, aluminum sulfate, sodium aluminum lactate, complexes of aluminum chlorohydrate and of glycol, such as the complex of aluminum chlorohydrate and of propylene glycol, the complex of aluminum dichlorohydrate and of propylene glycol, the complex of aluminum sesquichlorohydrate and of propylene glycol, the complex of aluminum chlorohydrate and of polyethylene glycol, the complex of aluminum dichlorohydrate and of polyethylene glycol, or the complex of aluminum sesquichlorohydrate and of polyethylene glycol.

As examples of sunscreens that can be combined with the glycolic extract (GE), obtained according to the preparation process as defined above, and present in the cosmetic formulations for topical use that are used in the process which is a subject of the present invention, mention may be made of all those which appear in the Cosmetics Directive 76/768/EEC, amended, Annex VII.

Among the organic sunscreens that can be combined with the glycolic extract (GE), obtained according to the preparation process as defined above, and present in the cosmetic formulations for topical use that are used in the process which is a subject of the present invention, mention may be made of the family of benzoic acid derivatives, such as para-aminobenzoic acids (PABAs), in particular monoglyceryl esters of PABA, ethyl esters of N,N-propoxy PABA, ethyl esters of N,N-diethoxy PABA, ethyl esters of N,N-dimethyl PABA, methyl esters of N,N-dimethyl PABA, butyl esters of N,N-dimethyl PABA; the family of anthranilic acid derivatives, such as homomenthyl-N-acetyl anthranilate; the family of salicylic acid derivatives, such as amyl salicylate, homomenthyl salicylate, ethylhexyl salicylate, phenyl salicylate, benzyl salicylate, p-isopropanolphenyl salicylate; the family of cinnamic acid derivatives, such as ethylhexyl cinnamate, ethyl-4-isopropyl cinnamate, methyl-2,5-diisopropyl cinnamate, p-methoxypropyl cinnamate, p-methoxyisopropyl cinnamate, p-methoxyisoamyl cinnamate, p-methoxyoctyl cinnamate (p-methoxy 2-ethylhexyl cinnamate), p-methoxy 2-ethoxyethyl cinnamate, p-methoxycyclohexyl cinnamate, ethyl-α-cyano-β-phenyl cinnamate, 2-ethylhexyl-α-cyano-β-phenyl cinnamate, glyceryl di-para-methoxy mono-2-ethylhexanoyl cinnamate; the family of benzophenone derivatives, such as 2,4-dihydroxybenzophenone, 2,2′-dihydroxy-4-methoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl-4′-phenylbenzophenone-2-carboxylate, 2-hydroxy-4-n-octyloxybenzophenone, 4-hydroxy-3-carboxybenzophenone; 3-(4′-methylbenzylidene)-d,l-camphor, 3-(benzylidene)-d,l-camphor, benzalkonium methosulfate camphor; urocanic acid, ethyl urocanate; the family of sulfonic acid derivatives, such as 2-phenylbenzimidazole 5-sulfonic acid and salts thereof; the family of triazine derivatives, such as hydroxyphenyltriazine, ethylhexyloxyhydroxyphenyl-4-methoxyphenyltriazine, 2,4,6-trianilino-(p-carbo-2′-ethylhexyl-1′-oxy)- 1,3,5-triazine, benzoic acid 4,4-((6-(((1,1- dimethylethyl)amino)carbonyl)phenyl)amino)-1,3,5-triazine-2,4-diyldiimino) bis(2-ethylhexyl) ester, 2-phenyl-5-methylbenzoxazole, 2,2′-hydroxy-5-methylphenylbenzotriazole, 2-(2′-hydroxy-5′-t-octylphenyl)benzotriazole, 2-(2′-hydroxy-5′-methyphenyl)benzotriazole; dibenzazine; dianisoylmethane, 4-methoxy-4″-t-butylbenzoylmethane; 5-(3,3-dimethyl-2-norbornylidene)-3-pentan-2-one; the family of diphenyl acrylate derivatives, such as 2-ethylhexyl-2-cyano-3,3-diphenyl-2-propenoate, ethyl-2-cyano-3,3-diphenyl-2-propenoate; the family of polysiloxanes, such as benzylidenesiloxane malonate; more particularly 2-(4-diethylamino-2-hydroxybenzoyl)benzoic acid hexyl ester, 2,4-bis{[4-(2-ethylhexyloxy)-2-hydroxy]phenyl}-6-(4-methoxyphenyl)-1,3,5-triazine, 2,4,6-tris[4-(2 ethylhexyloxycarbonyl)anilino]-1,3,5-triazine and 2-ethylhexyl dimethoxybenzylidene dioxoimidazolidine propionate.

Among the inorganic sunscreens, also called “mineral screens”, that can be combined with the glycolic extract (GE), obtained according to the preparation process as defined above, and present in the cosmetic formulations for topical use that are used in the process which is a subject of the present invention, mention may be made of titanium oxides, zinc oxides, cerium oxide, zirconium oxide, yellow, red or black iron oxides, and chromium oxides. These mineral screens may or may not be micronized, may or may not have undergone surface treatments and may be optionally provided in the form of aqueous or oily predispersions.

As examples of thickening and/or gelling surfactants that can be combined with the glycolic extract (GE), obtained according to the preparation process as defined above, and present in the cosmetic formulations for topical use that are used in the process which is a subject of the present invention, mention may be made of optionally alkoxylated alkylpolyglycoside fatty esters, for instance ethoxylated methylpolyglucoside esters, such as the PEG 120 methyl glucose trioleate and the PEG 120 methyl glucose dioleate sold respectively under the names Glucamate™ LT and Glucamate™ DOE120; alkoxylated fatty esters, such as the PEG 150 pentaerythrytyl tetrastearate sold under the name Crothix™ DS53, the PEG 55 propylene glycol oleate sold under the name Antil™ 141; fatty-chain polyalkylene glycol carbamates , for instance the PPG-14 laureth isophoryl dicarbamate sold under the name Elfacos™ T211, the PPG-14 palmeth-60 hexyl dicarbamate sold under the name Elfacos™ GT2125.

As examples of thickeners and/or gelling agents that can be combined with the glycolic extract (GE), obtained according to the preparation process as defined above, and present in the cosmetic formulations for topical use that are used in the process which is a subject of the present invention, mention may be made of linear or branched or crosslinked polymers of polyelectrolyte type, such as the partially or totally salified acrylic acid homopolymer, the partially or totally salified methacrylic acid homopolymer, the partially or totally salified 2-methyl-[(1-oxo-2-propenyl)amino]-1-propanesulfonic acid (AMPS) homopolymer, copolymers of acrylic acid and of AMPS, copolymers of acrylamide and of

AMPS, copolymers of vinylpyrrolidone and of AMPS, copolymers of AMPS and of (2-hydroxyethyl) acrylate, copolymers of AMPS and of (2-hydroxyethyl) methacrylate, copolymers of AMPS and of hydroxyethylacrylamide, copolymers of AMPS and of N,N-dimethylacrylamide, copolymers of AMPS and of tris(hydroxymethyl)acrylamidomethane (THAM), copolymers of acrylic or methacrylic acid and of (2-hydroxyethyl) acrylate, copolymers of acrylic or methacrylic acid and of (2-hydroxyethyl) methacrylate, copolymers of acrylic or methacrylic acid and of hydroxyethylacrylamide, copolymers of acrylic or methacrylic acid and of THAM, copolymers of acrylic or methacrylic acid and of N,N-dimethylacrylamide, terpolymers of acrylic or methacrylic acid, of AMPS and of (2-hydroxyethyl) acrylate, terpolymers of acrylic or methacrylic acid, of AMPS and of (2-hydroxyethyl) methacrylate, terpolymers of acrylic or methacrylic acid, of AMPS and of THAM, terpolymers of acrylic or methacrylic acid, of AMPS and of N,N-dimethylacrylamide, terpolymers of acrylic or methacrylic acid, of AMPS and of acrylamide, copolymers of acrylic acid or methacrylic acid and of alkyl acrylates, the carbon chain of which comprises between 4 and 30 carbon atoms and more particularly between 10 and 30 carbon atoms, copolymers of AMPS and of alkyl acrylates, the carbon chain of which comprises between 4 and 30 carbon atoms and more particularly between 10 and 30 carbon atoms, linear, branched or crosslinked terpolymers of at least one monomer having a free, partially salified or totally salified strong acid function, with at least one neutral monomer, and at least one monomer of formula (I):

CH₂═C(R′₃)—C(═O)—[CH₂—CH₂—O]ₙ—R′₄ (I)

wherein R′₃ represents a hydrogen atom or a methyl radical, R′₄ represents a linear or branched alkyl radical comprising from 8 to 30 carbon atoms and n represents a number greater than or equal to one and less than or equal to 50.

The linear or branched or crosslinked polymers of polyelectrolyte type that can be combined with the glycolic extract (GE), obtained according to the preparation process as defined above, and present in the cosmetic formulations for topical use that are used in the process which is a subject of the present invention, can be in the form of a solution, an aqueous suspension, a water-in-oil emulsion, an oil-in-water emulsion or a powder. The linear or branched or crosslinked polymers of polyelectrolyte type that can be combined with the glycolic extract (GE), obtained according to the preparation process as defined above, and present in the cosmetic formulations for topical use that are used in the process which is a subject of the present invention, can be selected from the products sold under the names SIMULGEL™ EG, Simulgel™EPG, Sepigel™ 305, Simulgel™ 600, Simulgel™ NS, Simulgel™ INS 100, Simulgel™ FL, Simulgel™ A, Simulgel™ SMS 88, Sepinov™EMT 10, Sepiplus™ 400, Sepiplus™265, Sepiplus™S, Sepimax™Zen, Aristoflex™AVC, Aristoflex™AVS, Novemer™EC-1, Novemer™EC 2, Aristoflex™HMB, Cosmedia™SP, Flocare™ET 25, Flocare™ET 75, Flocare™ET 26, Flocare™ET 30, Flocare™ET 58, Flocare™PSD 30, Viscolam™AT 64, Viscolam™AT 100.

As examples of thickeners and/or gelling agents that can be combined with the glycolic extract (GE), obtained according to the preparation process as defined above, and present in the cosmetic formulations for topical use that are used in the process which is a subject of the present invention, mention may be made of polysaccharides consisting only of monosaccharides, such as glucans or glucose homopolymers, glucomannoglucans, xyloglycans, galactomannans of which the degree of substitution (DS) of the D-galactose units on the main D-mannose chain is between 0 and 1, and more particularly between 1 and 0.25, such as galactomannans originating from cassia gum (DS=⅕), locust bean gum (DS=¼), tara gum (DS=⅓), guar gum (DS=½) or fenugreek gum (DS=1).

As examples of thickeners and/or gelling agents that can be combined with the glycolic extract (GE), obtained according to the preparation process as defined above, and present in the cosmetic formulations for topical use that are used in the process which is a subject of the present invention, mention may be made of polysaccharides consisting of monosaccharide derivatives, such as sulfated galactans and more particularly carrageenans and agar, uronans and more particularly algins, alginates and pectins, heteropolymers of monosaccharides and uronic acids, and more particularly xanthan gum, gellan gum, gum arabic exudates and karaya gum exudates, glucosaminoglycans.

As examples of thickeners and/or gelling agents that can be combined with the glycolic extract (GE), obtained according to the preparation process as defined above, and present in the cosmetic formulations for topical use that are used in the process which is a subject of the present invention, mention may be made of cellulose, cellulose derivatives, such as methylcellulose, ethylcellulose or hydroxypropylcellulose, silicates, starch, hydrophilic starch derivatives, and polyurethanes.

As examples of stabilizers that can be combined with the glycolic extract (GE), obtained according to the preparation process as defined above, and present in the cosmetic formulations for topical use that are used in the process which is a subject of the present invention, mention may be made of microcrystalline waxes, and more particularly ozokerite, mineral salts such as sodium chloride or magnesium chloride, and silicone polymers such as polysiloxane polyalkyl polyether copolymers.

As examples of spring or mineral waters that can be combined with the glycolic extract (GE), obtained according to the preparation process as defined above, and present in the cosmetic formulations for topical use that are used in the process which is a subject of the present invention, mention may be made of spring or mineral waters having a mineralization of at least 300 mg/l, in particular Avene water, Vittel water, Vichy basin water, Uriage water, La Roche Posay water, La Bourboule water, Enghien-les-bains water, Saint-Gervais-les-bains water, Néris-les-bains water, Allevard-les-bains water, Digne water, Maizieres water, Neyrac-les-bains water, Lons le Saunier water, Rochefort water, Saint Christau water, Fumades water and Tercis-les-bains water.

As examples of hydrotropic agents that can be combined with the glycolic extract

(GE), obtained according to the preparation process as defined above, and present in the cosmetic formulations for topical use that are used in the process which is a subject of the present invention, mention may be made of xylene sulfonates, cumene sulfonates, n-hexyl polyglucoside, (2-ethylhexyl) polyglucoside or n-heptyl polyglucoside.

As examples of emulsifying surfactants that can be combined with the glycolic extract (GE), obtained according to the preparation process as defined above, and present in the cosmetic formulations for topical use that are used in the process which is a subject of the present invention, mention may be made of nonionic surfactants, anionic surfactants and cationic surfactants.

As examples of emulsifying nonionic surfactants that can be combined with the glycolic extract (GE), obtained according to the preparation process as defined above, and present in the cosmetic formulations for topical use that are used in the process which is a subject of the present invention, mention may be made of fatty acid esters of sorbitol, such as the products sold under the names Montane™40, Montane™60, Montane™70, Montane™80 and Montane™85; compositions comprising glyceryl stearate and ethoxylated stearic acid containing between 5 mol and 150 mol of ethylene oxide, such as the composition comprising ethoxylated stearic acid containing 135 mol of ethylene oxide and of glyceryl stearate, sold under the name Simulsol™ 165; mannitan esters; ethoxylated mannitan esters; sucrose esters; methyl glucoside esters; alkyl polyglycosides comprising a linear or branched, saturated or unsaturated aliphatic radical, and comprising from 14 to 36 carbon atoms, such as tetradecyl polyglucoside, hexadecyl polyglucoside, octadecyl polyglucoside, hexadecyl polyxyloside, octadecyl polyxyloside, eicosyl polyglucoside, dodecosyl polyglucoside, (2-octyldodecyl) polyxyloside, (12-hydroxystearyl) polyglucoside; compositions of linear or branched, saturated or unsaturated fatty alcohols, comprising from 14 to 36 carbon atoms, and of alkyl polyglycosides as described above, for example the compositions sold under the brand names Montanov™68, Montanov™14, Montanov™82, Montanov™202, Montanov™S, Montanov™WO18, Montanov™L, Fluidanov™20X and Easynov™.

As examples of anionic surfactants that can be combined with the glycolic extract (GE), obtained according to the preparation process as defined above, and present in the cosmetic formulations for topical use that are used in the process which is a subject of the present invention, mention may be made of glyceryl stearate citrate, cetearyl sulfate, soaps such as sodium stearate or triethanolammonium stearate, salified N-acylated derivatives of amino acids, such as for example stearoyl glutamate.

As examples of emulsifying cationic surfactants that can be combined with the glycolic extract (GE), obtained according to the preparation process as defined above, and present in the cosmetic formulations for topical use that are used in the process which is a subject of the present invention, mention may be made of amine oxides, quaternium-82 and the surfactants described in patent application WO96/00719 and mainly those of which the fatty chain comprises at least 16 carbon atoms.

As examples of opacifiers and/or pearlescent agents that can be combined with the glycolic extract (GE), obtained according to the preparation process as defined above, and present in the cosmetic formulations for topical use that are used in the process which is a subject of the present invention, mention may be made of sodium palmitate, sodium stearate, sodium hydroxystearate, magnesium palmitate, magnesium stearate, magnesium hydroxystearate, ethylene glycol monostearate, ethylene glycol distearate, polyethylene glycol monostearate, polyethylene glycol distearate, and fatty alcohols comprising from 12 to 22 carbon atoms.

As examples of oils that can be combined with the glycolic extract (GE), obtained according to the preparation process as defined above, and present in the cosmetic formulations for topical use that are used in the process which is a subject of the present invention, mention may be made of mineral oils such as liquid paraffin, liquid petroleum jelly, isoparaffins or white mineral oils; oils of animal origin, such as squalene or squalane; vegetable oils, such as phytosqualane, sweet almond oil, coconut oil, castor oil, jojoba oil, olive oil, rapeseed oil, groundnut oil, sunflower oil, wheat germ oil, corn germ oil, soybean oil, cottonseed oil, coriander oil, beechnut oil, alfalfa oil, poppy oil, pumpkin oil, evening primrose oil, millet oil, barley oil, rye oil, safflower oil, candlenut oil, passionflower oil, hazelnut oil, palm oil, shea butter, apricot kernel oil, beauty-leaf oil, sysymbrium oil, avocado oil, calendula oil, oils derived from flowers or vegetables, ethoxylated vegetable oils; synthetic oils, for instance fatty acid esters, such as butyl myristate, propyl myristate, isopropyl myristate, cetyl myristate, isopropyl palmitate, octyl palmitate, butyl stearate, hexadecyl stearate, isopropyl stearate, octyl stearate, isocetyl stearate, dodecyl oleate, hexyl laurate, propylene glycol dicaprylate, esters derived from lanolic acid, such as isopropyl lanolate or isocetyl lanolate, fatty acid monoglycerides, diglycerides and triglycerides, such as glyceryl triheptanoate, alkyl benzoates, hydrogenated oils, poly(alpha-olefin)s, polyolefins such as poly(isobutane), synthetic isoalkanes such as isohexadecane, isododecane, perfluoro oils; silicone oils such as dimethylpolysiloxanes, methylphenylpolysiloxanes, amine-modified silicones, fatty acid-modified silicones, alcohol-modified silicones, silicones modified with alcohols and fatty acids, silicones modified with polyether groups, epoxy-modified silicones, silicones modified with fluoro groups, cyclic silicones and silicones modified with alkyl groups. In the present application, the term “oils” is intended to mean compounds and/or mixtures of compounds which are water-insoluble, and which have a liquid appearance at a temperature of 25° C.

As examples of waxes that can be combined with the glycolic extract (GE), obtained according to the preparation process as defined above, and present in the cosmetic formulations for topical use that are used in the process which is a subject of the present invention, mention may be made of beeswax, carnauba wax, candelilla wax, ouricury wax, Japan wax, cork fiber wax, sugarcane wax, paraffin waxes, lignite waxes, microcrystalline waxes, lanolin wax; ozokerite; polyethylene wax; silicone waxes; vegetable waxes; fatty alcohols and fatty acids that are solid at ambient temperature; glycerides that are solid at ambient temperature. In the present application, the term “waxes” is intended to mean compounds and/or mixtures of compounds which are water-insoluble, and which have a solid appearance at a temperature of greater than or equal to 45° C.

A subject of the invention is also a glycolic extract (GE) of a unialgal biomass of small multicellular macroalga cells originating from the class Florideophyceae and obtained by the process comprising the following successive steps:
a step A) of preparing a unialgal sample of multicellular macroalga cells from a sample of macroalgae originating from the class Florideophyceae and taken from the natural environment;
a step B) of culturing said unialgal sample of multicellular macroalga cells obtained in step A) in seawater supplemented with at least one nitrogen source, so as to obtain an aqueous suspension of said unialgal biomass of small multicellular macroalga cells;
a step C) of harvesting said unialgal biomass of small multicellular macroalga cells from said aqueous suspension obtained at the end of step B);
optionally, a step D) of preparing a powder of said unialgal biomass of small multicellular macroalga cells obtained in step C);
a step E) during which said unialgal biomass of small multicellular macroalga cells obtained in step C) or the powder of said biomass in step D) is dispersed with stirring in a water-glycol mixture in a proportion of from 1% by weight to 20% by weight of biomass per 100% by weight of dispersion; and
a step F) during which the dispersion obtained in the preceding step E) is separated into its immiscible phases, in order to collect said glycolic extract (GE), for use thereof in a method for therapeutic treatment of irritation of the skin, the scalp or the mucous membranes, redness, tingling and/or itching sensations, signs of degradation of the hair, an increase in the porosity thereof, the dulling thereof, and/or the weakening thereof with respect to mechanical stresses; and more particularly the glycolic extract (GE) of a unialgal biomass of cells of red algae originating from the class Florideophyceae and in the subclass Nemaliophycidae, and even more particularly the glycolic extract (GE) of a unialgal biomass of cells of red algae originating from the class Florideophyceae, the subclass Nemaliophycidae, order Acrochaetiales, family Acrochatiaceae, genus Acrochaetium and species Acrochaetium moniliforme.

The following examples illustrate the invention without, however, limiting it.

1—Evaluation of the Effect of a Glycolic Extract (in 1,3-Butanediol) of a Unialgal Biomass of Small Multicellular Macroalga Cells Originating from the Alga Acrochaetium moniliforme, called “Extract A”, on a Culture of Keratinocytes in the Presence of an Atmospheric Polluting Agent (Cadmium)

The extract A is obtained by carrying out the process for preparing the glycolic extract (GE) as described above, for which:
in step A) of the process for preparing the glycolic extract (GE), as described above, the red macroalga, taken from the natural environment, namely from seawater in the English Channel on the shore of the Côtes d'Armor, is the alga Acrochaetium moniliforme,
in step E) of the process for preparing the glycolic extract (GE), the glycol used is 1,3-butanediol, in a proportion of 40% by weight per 100% of the weight of the dispersion.

Principle of the evaluation: evaluation of the effects of an exposure to cadmium on a keratinocyte culture.

Protocol: Human keratinocytes are cultured in a 96-well culture plate. After a few days of culture, the products evaluated, namely the Extract A at 0.5% by weight and sulforaphane (a positive reference known to those skilled in the art), are applied in the culture medium. At the end of incubation for 24 hours, the cells are exposed to a solution of cadmium at 35 μM for 24 hours, then 24 hours later, the following various evaluations are carried out:
evaluation of the cell viability by means of a DNA assay by the Hoechst method
evaluation of the cell metabolism by means of a protein assay by the BCA method;
evaluation of the inflammatory component by means of an assay for the cytokines IL-1a and IL-8.

Results: The results relating to cell viability for the products evaluated are reported in table 1 below:

TABLE 1
evaluation of cell viability for the
Extract A) according to the invention

| | Mean values of the | Associated |
|---|---|---|
| | expressed proteins | standard deviation |
| Products tested | (μg/ml) | (μg/ml) |
| Control cells (non-polluted, non- | 300.7 | 5.56 |
| treated) | | |
| Polluted, non-treated cells | 65.2 | 5.33 |
| Polluted cells associated with | 156.7 | 25.25 |
| sulforaphane at 1 μM | | |
| Polluted cells associated with | 233.2 | 15.6 |
| the Extract A) at 0.5% | | |

The results relating to the evaluation of cell metabolism for the products evaluated are reported in table 2 below:

TABLE 2
evaluation of cell metabolism for the
Extract A) according to the invention

| | Mean values of the | Associated |
|---|---|---|
| | expressed DNA | standard deviation |
| Products tested | (μg/ml) | (μg/ml) |
| Control cells (non-polluted, non- | 25.2 | 1.52 |
| treated) | | |
| Polluted, non-treated cells | 2.54 | 0.44 |
| Polluted cells associated with | 6.7 | 1.75 |
| sulforaphane at 1 μM | | |
| Polluted cells associated with | 10.25 | 1.71 |
| the Extract A) at 0.5% | | |

The results relating to the evaluation of the inflammatory responses with regard to the cytokines IL-1α and IL-8 for the products evaluated are reported in table 3 below:

TABLE 3
evaluation of the inflammatory responses with regard to the
cytokine IL-1α for the Extract A) according to the invention

| | Mean values | Associated |
|---|---|---|
| | of IL-1α | standard |
| | expressed | deviation |
| | (pg/μg of | (pg/μg of |
| Products tested | proteins) | proteins) |
| Control cells (non-polluted, non-treated) | 0.2735 | 0.0389 |
| Polluted, non-treated cells | 6.7843 | 0.8304 |
| Polluted cells associated with sulforaphane | 0.8238 | 0.3765 |
| at 1 μM | | |
| Polluted cells associated with the Extract A) | 0.2831 | 0.0995 |
| at 0.5% | | |

Evaluation of the inflammatory responses with regard to the
cytokine IL-8 for the Extract A) according to the invention

| | Mean values | Associated |
|---|---|---|
| | of IL-8 | standard |
| | expressed | deviation |
| | (pg/μg of | (pg/μg of |
| Products tested | proteins) | proteins) |
| Control cells (non-polluted, non-treated) | 0.4499 | 0.1235 |
| Polluted, non-treated cells | 6.6947 | 0.9727 |
| Polluted cells associated with sulforaphane | 3.7081 | 0.6135 |
| at 1 μM | | |
| Polluted cells associated with the Extract A) | 2.5154 | 0.1850 |
| at 0.5% | | |

The application of cadmium in the culture medium leads to a decrease in the mean value of expressed proteins of from 300.7 μg/ml to 65.2 μg/ml (see table 1), that is to say a decrease of 78.2%, whereas the application of the same amount of cadmium in the culture medium comprising 0.5% by weight of the Extract A) results in a mean value of expressed protein of 233.2 μg/ml (see table 1), that is to say a decrease of only 22.4%. This protective effect is statistically significant, according to the Student's test (with p<0.001) compared with the control condition (“Cells polluted with cadmium and not treated”).

The application of cadmium in the culture medium leads to a decrease in the mean value of the amount of DNA produced by the cells of from 25.12 μg/ml to 2.54 μg/ml (see table 2), that is to say a decrease of 90.3%, whereas the application of the same amount of cadmium in the culture medium comprising 0.5% by weight of the Extract A) results in a mean value of DNA produced of 10.25 μg/ml (see table 2), that is to say a decrease limited to 58.9%. This protective effect is statistically significant, according to the Student's test (with p<0.01) compared with the control condition (“Cells polluted with cadmium and not treated”).

The application of cadmium in the culture medium leads to an increase in the mean value of interleukin IL-8 produced of from 0.4499 pg/μg (picogram/microgram) to 6.6947 pg/μg (see table 3), that is to say an increase of 1388%, whereas the application of the same amount of cadmium in the culture medium comprising 0.5% by weight of the Extract A) results in a mean value of interleukin IL-8 produced of 2.5154 pg/μg (see table 3), that is to say an increase of 459%. This protective effect is statistically significant, according to the Student's test (with p<0.01) compared with the control condition (“Cells polluted with cadmium and not treated”).

The application of cadmium in the culture medium leads to an increase in the mean value of interleukin IL-1α produced of from 0.2735 pg/μg to 6.7843 pg/μg (see table 3), that is to say an increase of 2380%, whereas the application of the same amount of cadmium in the culture medium comprising 0.5% by weight of the Extract A) results in a mean value of interleukin IL-1α produced of 0.2831, that is to say an increase of 3.5%. This protective effect is statistically significant, according to the Student's test (with p<0.001) compared with the control condition (“Cells polluted with cadmium and not treated”).

It can therefore be concluded from these experimental observations that Extract A), used in the process according to the invention, is characterized by a protective effect on the cells of human skin subjected to exposure to a polluting agent, by protecting the metabolism of said cells and the integrity (viability) thereof and by limiting the inflammation phenomena, and therefore the consequences thereof on the skin, the mucous membranes, the hair and the scalp.

2—Evaluation of the Effect of a Glycolic Extract (in 1,3-Butanediol) of a Unialgal Biomass of Small Multicellular Macroalga Cells Originating from the Alga Acrochaetium moniliforme, Called “Extract A”, on Patients Subjected to an Ozone-Rich Polluted Atmosphere

2.1 Principle of the Evaluation:

Squalene is a lipid specific to human sebum which, because of its chemical structure and in particular the presence of ethylenic bonds, acts as an antioxidant barrier at the surface of the skin, but is very sensitive to oxidation phenomena, and more particularly to those involving ozone as oxidizing reagent. It is thus known in the literature (A. Whisthaler and C. J. Weschler, PNAS, Apr. 13, 2010; Vol. 107, no.15, pp 6568-6575, “Reaction of ozone with human skin lipids: sources of carbonyls, dicarbonyls, and hydroxycarbonyls in indoor air”), that ozonolysis of squalene results in the generation of numerous degradation products, the main 3 of which are C27-pentaenal, C22-tetraenal and C17-trienal, which, at the end of an ozonolysis process of which they are themselves the subject, generate volatile products such as, for example, acetone, 6-methyl-5-hepten-2-one, 2,6-dimethyl-2,6-undecandien-10-one (geranyl acetone), 1-hydroxypropan-2-one (hydroxy acetone); said volatile products, in particular the dicarbonylated species, are respiratory irritants and human skin irritants (A. Whisthaler and C. J. Weschler, PNAS, Apr. 13, 2010 ; Vol. 107, no.15, pp 6568-6575, “Reaction of ozone with human skin lipids: sources of carbonyls, dicarbonyls, and hydroxycarbonyls in indoor air”).

The principle of the evaluation of the Extract A on patients subjected to an ozone-rich polluted atmosphere therefore lies in measuring the change over time of the cumulative content of C27-pentaenal, of C22-tetraenal and of C17-trienal that are present in the sebum of the patients subjected to a “polluted” atmosphere characterized by its higher than average ozone content.

2.2 Protocol:

The study was carried out in Shanghai in autumn 2015, under proven pollution conditions measured according to the AQI (Air Quality Index) international standard of “moderate level” to “unhealthy”. In terms of ozone content, the values were measured between 6 μg/m³ and 183 μg/m³. A group of 20 volunteers was recruited in order to successfully bring this study to conclusion.

Characteristics of the panel: Chinese women, 25 to 60 years old (and before the signs of the menopause), having an established oily skin, with inclusion criterion at 140 μg/cm² on the forehead measured with a Sebumeter® (sold by the company Courage & Khazaka) at the beginning of the study, and/or having skin with imperfections and open pores.

The study lasted two months, and consisted in preparing an oil-in-water emulsion of which the composition and the weight content of each of the ingredients, for 100% of the weight thereof, are reported in table 4 below:

TABLE 4
oil-in-water emulsion tested

| Ingredients | Weight content (%) |
|---|---|
| Water | qs for 100 |
| Glycerol | 3 |
| Solagum AX⁽¹⁾ | 0.3 |
| Montanov 202⁽²⁾ | 2 |
| Lanol 99⁽³⁾ | 7 |
| Cetiol OE⁽⁴⁾ | 3 |
| Lanol P⁽⁵⁾ | 0.25 |
| Sepiplus 400⁽⁶⁾ | 0.8 |
| Euxyl PE9010⁽⁷⁾ | 1 |
| Euxyl K900⁽⁸⁾ | 0.5 |
| Extract A | 1 |

⁽¹⁾Solagum ™ AX (INCI name: Acacia Senegal gum & xanthan gum) is an emulsifier;
⁽²⁾Montanov ™202 (INCI name: Arachidyl alcohol & behenyl alcohol & arachidyl glucoside) is an emulsifier;
⁽³⁾Lanol ™99 (INCI name: Isononyl isononanoate) is an emollient ester;
⁽⁴⁾Cetiol ™ OE (INCI name: Dicaprylyl ether) is a cosmetic fatty phase;
⁽⁵⁾Lanol ™ P (INCI name: Glycol palmitate) is a cosmetic fatty phase;
⁽⁶⁾Sepiplus ™400 (INCI name: Polyacrylate 13 & polysorbate 20 & polyisobutene & sorbitan isostearate) is a thickening, emulsifying and stabilizing agent, in the form of an inverse latex;
⁽⁷⁾Euxyl ™ PE9010 (INCI name: Phenoxyethanol & ethylhexylglycerin) is a preservative;
⁽⁸⁾Euxyl ™ K900 (INCI name: Benzyl Alcohol & Ethylhexylglycerin & Tocopherol) is a preservative.

Said emulsion was administered to the group of 20 individuals as described above for 56 days, by twice daily application. Sebum samples were taken from the forehead of the volunteers on DO (beginning of the study) and D56 (end of the study). The analysis of said samples consisted in searching for and assaying the 3 compounds initially resulting from the ozonolysis of squalene, namely C27-pentaenal, C22-tetraenal and C17-trienal, by carrying out an analytical method involving gas chromatography-mass spectrometry; the cumulative content of these three compounds reflecting the level of intensity of the effects of the pollution on the skin.

2.3 Expression of the Results:

The quantification of each of the 3 compounds initially resulting from the ozonolysis of squalene, namely C27-pentaenal, C22-tetraenal and C17-trienal, is carried out for each sample on DO and on D56, and the sum of these 3 contents is called “ozonolyzed squalene” (O.S). The mean variation observed (Δ%) after two months of use of the product was thus evaluated, and expressed as percentage according to the formula:

Δ%=100×(O.S. D₅₆−O.S. O.S. D₀)/O.S. D₀,

with O.S.: ozonolyzed squalene content; J₀: 1st day of the study; D56: fifty-sixth day of the study.

2.4 Results: the variation in the ozonolyzed squalene (Δ%), measured for the volunteers having applied the composition detailed in table 4, is established at −61% and is statistically significant according to the Student's test (p<0.001, t-test).

This experimental result thus shows a significant protective action inherent in the application of the composition comprising the Extract A) for skin subjected to a polluting agent, in the case in point ozone.

3-Illustration Formulae

3.1. Organo-Mineral, Anti-Pollution Sun Spray

### FORMULA

| | | |
|---|---|---|
| A | Isodecyl neopentanoate | 20% |
| | Cyclodimethicone | 5% |
| | Ethylhexylmethoxycinnamate | 6% |
| | Butyl Methoxydibenzoylmethane | 3% |
| | DL-alpha-Tocopherol | 0.05% |
| B | Water | qs for 100% |
| | Tetrasodium EDTA | 0.2% |
| | Glycerol | 7% |
| | Phenyl Benzimidazole Sulfonic Acid | 3% |
| | (salified with required molar amount | |
| | of sodium hydroxide) | |
| C | SEPIMAX ™Zen | 1.3% |
| | Keltrol ™CG-T | 0.315% |
| | Efficacia ™M | 0.385% |
| D | Extract A | 1% |
| | SEPICIDE ™ HB | 1% |
| | Fragrance | 0.1% |

3.2: Anti-Pollution Face Mask Cream-Gel

### FORMULA

| | | |
|---|---|---|
| A | Simmondsia chinensis seed oil | 14.1% |
| | C₁₂-C₁₅ Alkyl benzoate | 6.7% |
| | Cyclopentasiloxane | 4.2% |
| | DL-alpha-Tocopherol | 0.10% |
| B | Maris Aqua | qs for 100% |
| C | SEPIMAX ™Zen | 2% |
| | Keltrol ™CG-T | 0.45% |
| | Efficacia ™M | 0.55% |
| D | Extract A | 1.2% |
| | Euxyl PE9010 | 1% |
| | Fragrance | 0.1% |

3.3: Antisun Water-in-Oil Emulsion

| | |
|---|---|
| Diisopropyl adipate | 12% |
| (Ethylhexyl) salicylate | 5% |
| (Ethylhexyl) methoxycinnamate | 5% |
| (Ethylhexyl) 4-(N,N-dimethylamino)benzoate | 8% |
| Butylmethoxydibenzoylmethane | 2% |
| PEG30 dipolyhydroxystearate | 0.4% |
| Fluidanov ™ 20X | 1.2% |
| Sepimax Zen | 0.7% |
| Glycerol: | 1.5% |
| Euxyl ™ PE9010: | 1% |
| Extract A) | 1% |
| Water: | qs for 100% |

3.4: Anti-Pollution Lightening Water-in-Oil Emulsion

| | |
|---|---|
| Isononyl isononanoate: | 6% |
| Fragrance: | 0.1% |
| Phenoxyethanol & (Ethylhexyl) Glycerin: | 1% |
| Easynov ™: | 1.5% |
| SEPIMAX ™Zen | 0.5% |
| Sepiwhite ™MSH: | 2% |
| Extract A) | 0.9% |
| Water: | qs for 100% |

3.5: Anti-Pollution Dyeing Shampoo

Formula

| | |
|---|---|
| Phase A | |
| MONTALINE ™C40 | 15% |
| Disodium cocoamphoacetate | 5% |
| Cetrimonium chloride | 1% |
| MONTANOV ™S | 3% |
| SEPIMAX ™ Zen | 1.3% |
| Phase B | |
| Extract A) | 1% |
| Color | qs |
| Water | qs for 100% |

3.6: Anti-Pollution Hair Lotion

| | |
|---|---|
| Butylene glycol | 3.0% |
| Sepimax ™ Zen | 3.0% |
| SIMULSOL ™1293 | 3.0% |
| Lactic acid | qs pH = 6 |
| SEPICIDE ™ HB | 0.2% |
| Extract A) | 1.3% |
| Fragrance | 0.3% |
| Water | qs for 100% |

SEPIMAX™Zen (INCI name: Polyacrylate Crosspolymer-6) is a thickening, emulsifying and stabilizing agent;

Keltrol™CG-T: Xanthan gum;

Efficacia™M is the acacia gum sold by the company CNI;

SEPICIDE™ HB (INCI name: Phenoxyethanol/Methylparaben/Ethylparaben/Propylparaben/Butylparaben) is a preservative containing phenoxyethanol;

Euxyl PE9010 (INCI name: Phenoxyethanol & Ethylhexyl Glycerin): composition used as a preservative;

Maris Aqua: seawater containing 8% sodium chloride;

Fluidanov™ 20X (INCI name: octyldodecanol and octyldodecyl xyloside) is a water-in-oil emulsifying composition;

Sepiwhite™MSH (INCI name: w-undecylenoyl phenylalanine) is a skin-lightening agent; MONTALINE™C40: (cocamoniumcarbamoyl chloride);

MONTANOV™S: (cocoyl glucoside & cocoyl alcohol);

SIMULSOL™1293 is hydrogenated and ethoxylated castor oil, with an ethoxylation index equal to 40.

1. A process for preventing or slowing the appearance, on the skin, the scalp, the hair or the mucous membranes, of unattractive signs generated by the polluting agents present in the atmosphere or else for eliminating said signs, said process comprising at least one step of applying, to human skin, to the mucous membranes, to the scalp or to the hair, a cosmetic formulation for topical use comprising at least one cosmetically acceptable excipient and an effective amount of a glycolic extract (GE) of a unialgal biomass of small multicellular macroalga cells originating from the class Florideophyceae, said extract being obtained by the process comprising the following successive steps:
a step A) of preparing a unialgal sample of multicellular macroalga cells from a sample of macroalgae originating from the class Florideophyceae and taken from the natural environment;
a step B) of culturing said unialgal sample of multicellular macroalga cells obtained in step A) in seawater supplemented with at least one nitrogen source, so as to obtain an aqueous suspension of said unialgal biomass of small multicellular macroalga cells;
a step C) of harvesting said unialgal biomass of small multicellular macroalga cells from said aqueous suspension obtained at the end of step B);
optionally, a step D) of preparing a powder of said unialgal biomass of small multicellular macroalga cells obtained in step C);
a step E) during which said unialgal biomass of small multicellular macroalga cells obtained in step C) or the powder of said biomass in step D) is dispersed with stirring in a water-glycol mixture in a proportion of from 1% by weight to 20% by weight of biomass per 100% by weight of dispersion; and
a step F) during which the dispersion obtained in the preceding step E) is separated into its immiscible phases, so as to collect said glycolic extract (GE).

2. The process as defined in claim 1, for which the glycol used in step (E) is 1, 3-butanediol.

3. The process as defined in claim 1, for which said unattractive signs generated by the polluting agents present in the atmosphere are redness, wrinkles, fine lines or degradation of the microrelief of the skin, of the scalp or of the mucous membranes, dulling of the skin complexion or else dulling of the hair and/or weakening thereof with respect to mechanical stresses.

4. The process as defined in claim 1, for which said small multicellular macroalga cells originating from the class Florideophyceae, constituting said unialgal biomass, originate from the subclass Nemaliophycidae, order Acrochaetiales, family Acrochatiaceae, genus Acrochaetium and species Acrochaetium moniliforme.

5. A glycolic extract (GE) of a unialgal biomass of small multicellular macroalga cells originating from the class Florideophyceae and obtained by the process comprising the following successive steps:
a step A) of preparing a unialgal sample of multicellular macroalga cells from a sample of macroalgae originating from the class Florideophyceae and taken from the natural environment;
a step B) of culturing said unialgal sample of multicellular macroalga cells obtained in step A) in seawater supplemented with at least one nitrogen source, so as to obtain an aqueous suspension of said unialgal biomass of small multicellular macroalga cells;
a step C) of harvesting said unialgal biomass of small multicellular macroalga cells from said aqueous suspension obtained at the end of step B);
optionally, a step D) of preparing a powder of said unialgal biomass of small multicellular macroalga cells obtained in step C);
a step E) during which said unialgal biomass of small multicellular macroalga cells obtained in step C) or the powder of said biomass in step D) is dispersed with stirring in a water-glycol mixture in a proportion of from 1% by weight to 20% by weight of biomass per 100% by weight of dispersion; and
a step F) during which the dispersion obtained in the preceding step E) is separated into its immiscible phases, so as to collect said glycolic extract (GE), for use thereof in a method for therapeutic treatment of irritation of the skin, of the scalp or of the mucous membranes, redness, tingling and/or itching sensations, signs of degradation of the hair, an increase in the porosity thereof, and the dulling thereof and/or the weakening thereof with respect to mechanical stresses.

6. The process as defined in claim 2, for which said unattractive signs generated by the polluting agents present in the atmosphere are redness, wrinkles, fine lines or degradation of the microrelief of the skin, of the scalp or of the mucous membranes, dulling of the skin complexion or else dulling of the hair and/or weakening thereof with respect to mechanical stresses.

7. The process as defined in claim 2, for which said small multicellular macroalga cells originating from the class Florideophyceae, constituting said unialgal biomass, originate from the subclass Nemaliophycidae, order Acrochaetiales, family Acrochatiaceae, genus Acrochaetium and species Acrochaetium moniliforme.

8. The process as defined in claim 3, for which said small multicellular macroalga cells originating from the class Florideophyceae, constituting said unialgal biomass, originate from the subclass Nemaliophycidae, order Acrochaetiales, family Acrochatiaceae, genus Acrochaetium and species Acrochaetium moniliforme.

9. The process as defined in claim 6, for which said small multicellular macroalga cells originating from the class Florideophyceae, constituting said unialgal biomass, originate from the subclass Nemaliophycidae, order Acrochaetiales, family Acrochatiaceae, genus Acrochaetium and species Acrochaetium moniliforme.

10. A process for preventing or slowing the appearance, on the skin, the scalp, the hair or the mucous membranes, of unattractive signs generated by the polluting agents present in the atmosphere or else for eliminating said signs, said process comprising at least one step of applying, to human skin, to the mucous membranes, to the scalp or to the hair, a cosmetic formulation for topical use comprising at least one cosmetically acceptable excipient and an effective amount of a glycolic extract (GE) of a unialgal biomass of small multicellular macroalga cells originating from the class Florideophyceae, said extract being obtained by the process comprising the following successive steps:
a step A) of preparing a unialgal sample of multicellular macroalga cells from a sample of macroalgae originating from the class Florideophyceae and taken from the natural environment;
a step B) of culturing said unialgal sample of multicellular macroalga cells obtained in step A) in seawater supplemented with at least one nitrogen source, so as to obtain an aqueous suspension of said unialgal biomass of small multicellular macroalga cells;
a step C) of harvesting said unialgal biomass of small multicellular macroalga cells from said aqueous suspension obtained at the end of step B);
a step D) during which said unialgal biomass of small multicellular macroalga cells obtained in step C) is dispersed with stirring in a water-glycol mixture in a proportion of from 1% by weight to 20% by weight of biomass per 100% by weight of dispersion; and
a step E) during which the dispersion obtained in the preceding step d) is separated into its immiscible phases, so as to collect said glycolic extract (GE).

11. The process of claim 10, wherein the step C) further comprises preparing a powder of said unialgal biomass of small multicellular macroalga cells, and step D) is performed using the powder.

12. The process as defined in claim 11, for which the glycol used in step (E) is 1,3-butanediol.

13. The process as defined in claim 11, for which said unattractive signs generated by the polluting agents present in the atmosphere are redness, wrinkles, fine lines or degradation of the microrelief of the skin, of the scalp or of the mucous membranes, dulling of the skin complexion or else dulling of the hair and/or weakening thereof with respect to mechanical stresses.

14. The process as defined in claim 12, for which said unattractive signs generated by the polluting agents present in the atmosphere are redness, wrinkles, fine lines or degradation of the microrelief of the skin, of the scalp or of the mucous membranes, dulling of the skin complexion or else dulling of the hair and/or weakening thereof with respect to mechanical stresses.

15. The process as defined in claim 11, for which said small multicellular macroalga cells originating from the class Florideophyceae, constituting said unialgal biomass, originate from the subclass Nemaliophycidae, order Acrochaetiales, family Acrochatiaceae, genus Acrochaetium and species Acrochaetium moniliforme.

16. The process as defined in claim 12, for which said small multicellular macroalga cells originating from the class Florideophyceae, constituting said unialgal biomass, originate from the subclass Nemaliophycidae, order Acrochaetiales, family Acrochatiaceae, genus Acrochaetium and species Acrochaetium moniliforme.

17. The process as defined in claim 13, for which said small multicellular macroalga cells originating from the class Florideophyceae, constituting said unialgal biomass, originate from the subclass Nemaliophycidae, order Acrochaetiales, family Acrochatiaceae, genus Acrochaetium and species Acrochaetium moniliforme.

18. The process as defined in claim 14, for which said small multicellular macroalga cells originating from the class Florideophyceae, constituting said unialgal biomass, originate from the subclass Nemaliophycidae, order Acrochaetiales, family Acrochatiaceae, genus Acrochaetium and species Acrochaetium moniliforme.
